(19) United States
(12) Patent Application Publication
(10) Pub. No.: US 2009/0182191 A1
(43) Pub. Date: Jul. 16, 2009

(54) Stoma Protective Cap

(76) Inventor: Hartmut Redlich, Mudau (DE)

Correspondence Address: Nixon Peabody LLP, 200 Page Mill Road, Suite 200, Palo Alto, CA, 94306, US

(21) Appl. No.: 12/350,963

(22) Filed: Jan. 9, 2009

(30) Foreign Application Priority Data
Jan. 11, 2008 (DE) 102008004174.2-51

(51) Int. Cl. A61F 2/82

(52) U.S. Cl. 600/32

(57) ABSTRACT

The stoma protective cap for protection of the stoma, encompassing a plate (4) which has a curvature (3), which curvature (3) is formed such that it spans a cavity across the stoma and the plate encompasses at least one retaining region (1) which enables a fastening on clothing so that the plate can be securely held between the clothing and the body.

### FIELD OF THE INVENTION

For medical care after installation of an anus (colostomy/ileostomy), a base plate is installed at the stoma, said base plate is connected by a closing ring to a bag which serves to accommodate stools.

It should be understood that the invention is not restricted to the anus, but rather can also be applied to urostomy care. This involves a catheter which in the same manner as the stoma is fitted with a base plate and a bag. The term stoma should be understood below for both applications and not considered in a restricted manner.

This medical care unit substantially comprises a base plate and a collection bag for stools, said base plate and collection bag are connected to a closing ring system. This closing ring system comprises a flexible adhesive plaster which is provided with a more stable closing ring to which the collection bag is clamped.

In order to protect this system from pressure, there exists in the sanitary offering a protection in combination with a truss, wherein the stoma bag is pulled through a hole in the truss and protected against pressure by a plastic cap which is fastened by a Velcro strip over the hole of the truss. In this context, reference is made to the documents (DE 299 00 295 U1, DE 1786 120, DE 1714036U, DD 39645A1) However, this approach brings with it a number of problems such as e.g. compression of the bag.

### OVERVIEW OF THE INVENTION

The object of the invention is to provide a simpler protective cap which offers significantly better protection.

This object is achieved by an arrangement or device with the features of the independent claims. The key problems are solved with the new stoma protective cap as described further below.

Herein, a cap is used which is preferably clamped onto the trouser waistband or a belt. In detail, the stoma protective cap involves a stable, skin-friendly plate. The plate has a curvature which is formed such that it spans a cavity across the stoma. As a result, direct contact of clothing or the belt with the stoma should be prevented. To ensure that the plate does not slip, it encompasses at least one retaining region which enables a fastening on clothing so that the plate is securely held between the clothing and the body. The retaining region can be of various types. A clamping region, hook-in regions, pressure button regions, Velcro regions or the like are thus conceivable.

In the preferred embodiment, there is a clamping region or a hook-in region which makes it possible to hook the stoma protective cap into clothing. Hooking in is preferably carried out onto the trouser waistband or behind a belt.

The hook-in region which is also referred to as a guide is formed as an elongated hole in a preferred form. Wherein the guides in the form of elongated holes run from the bottom to the top (herein the plate in the form used is considered). The lower region of the guide is formed open, wherein the upper region of the elongated hole ends in the stoma protective cap so that a pushing onto a trouser waistband or a belt from top to bottom is enabled. The trouser waistband or the belt are herein threaded into the guides in the form of elongated holes. If one views the stoma protective cap from left to right, the trouser waistband or the belt is guided below the stoma protective cap in the first region, after which it is guided through the elongated hole in front of the stoma protective cap via a curvature in order then to be guided back under the stoma protective cap through the second elongated hole.

In one alternative embodiment, the guides are formed as brackets which are arranged on the front side of the stoma protective cap and which allow clothing to be pushed under the brackets.

In a further embodiment, the stoma protective cap is fundamentally opened downwards, wherein, to this end, the curvature forms an opening to the bottom so that the bag has sufficient space to the bottom.

As a result of this approach, one achieves certain advantages. These advantages encompass:

1.) No abdominal belt (truss) is required since the stoma protective cap can also be fastened on or at the normal stoma belt or on underpants with its lateral guide slots. A belt produced specifically for this purpose can also be used.

2.) The stoma bag is not compressed by the size of the stoma protective cap and the stoma can allow stools to escape unhindered under the stoma protective cap into the bag.

3.) The stoma protective cap can also be pushed with the lateral guide slots under the trouser waistband or skirt waistband so that the stoma protective cap is below the underclothes or also the tucked-in shirt or blouse. It is thus prevented that pressure is exerted on the stoma by clothing. Moreover, no chafing on the stoma occurs when walking.

The figures are briefly described below:

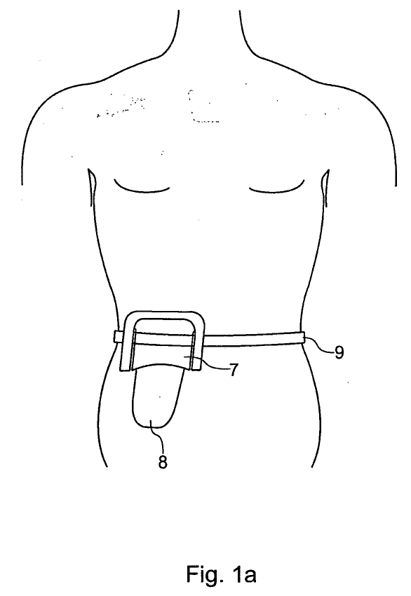

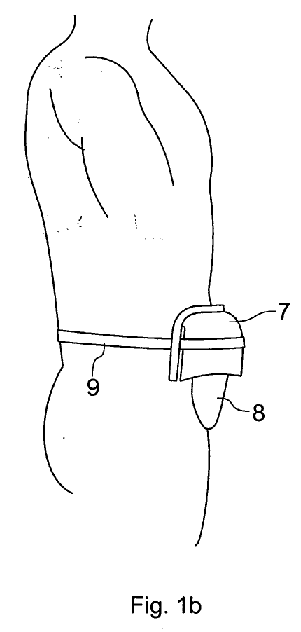

FIGS. 1a-1b show a stoma protective cap which is held on the body by a belt.

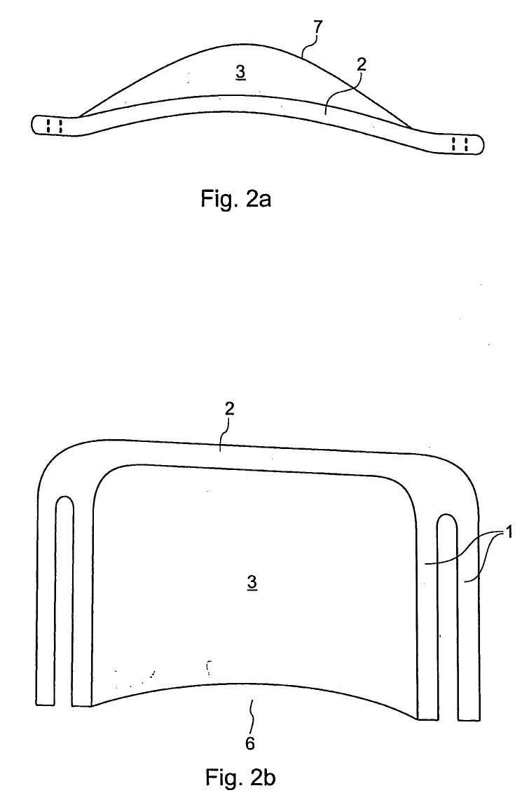

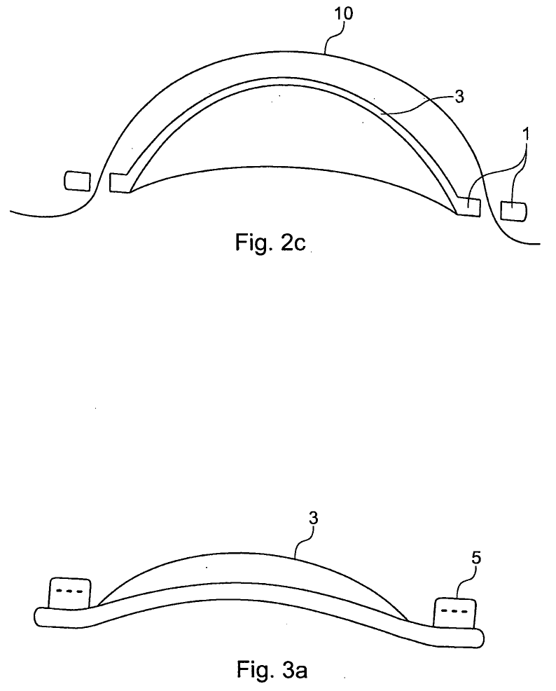

FIGS. 2a-2c show the stoma protective cap with guides in the form of an elongated hole from above, from the front and from below.

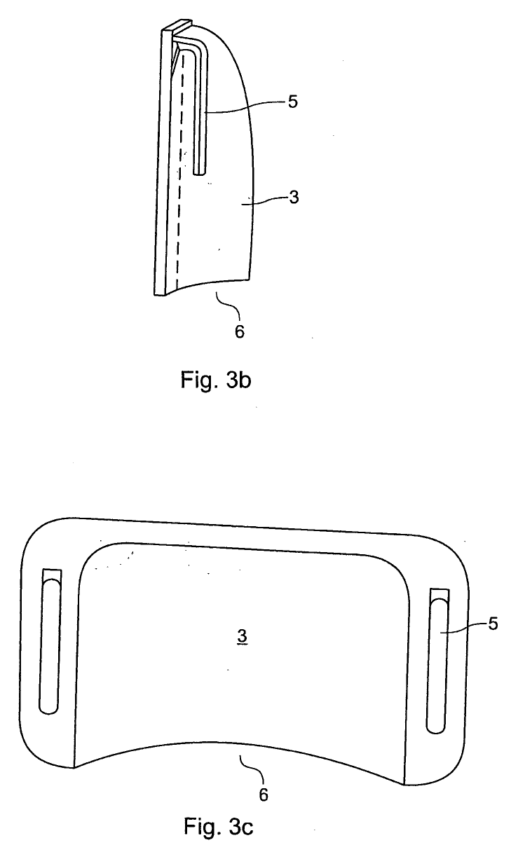

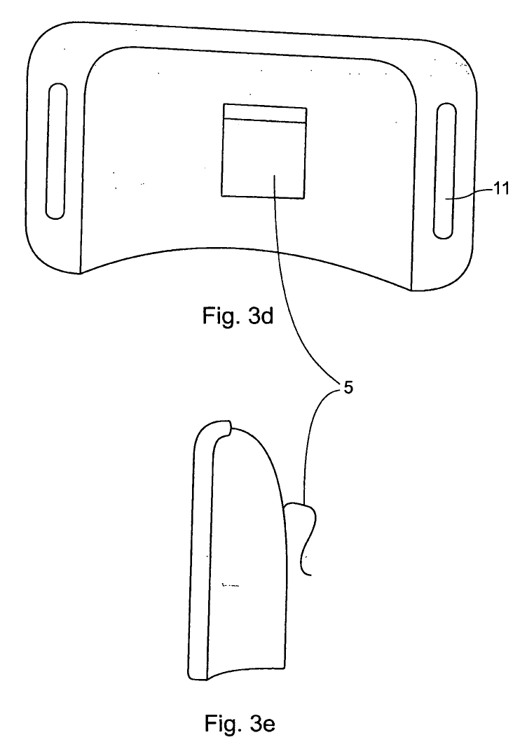

FIGS. 3a-3c show the stoma protective cap with a bracket from above, from the side and from the front.

FIGS. 3d-e show the stoma protective cap with a bracket wherein the bracket is formed at an alternative position so that it can be fastened onto commercially available stoma belts.

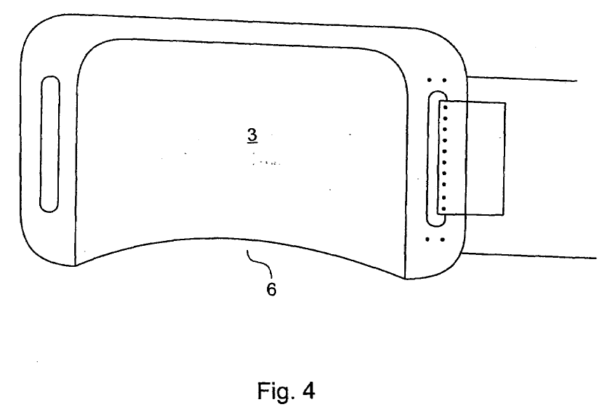

FIG. 4 shows a fastening of a stoma belt on the protective cap.

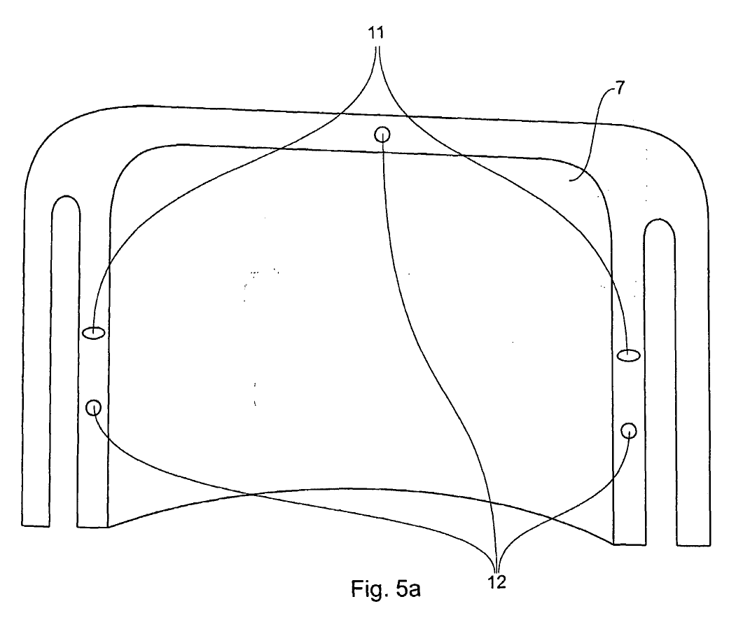

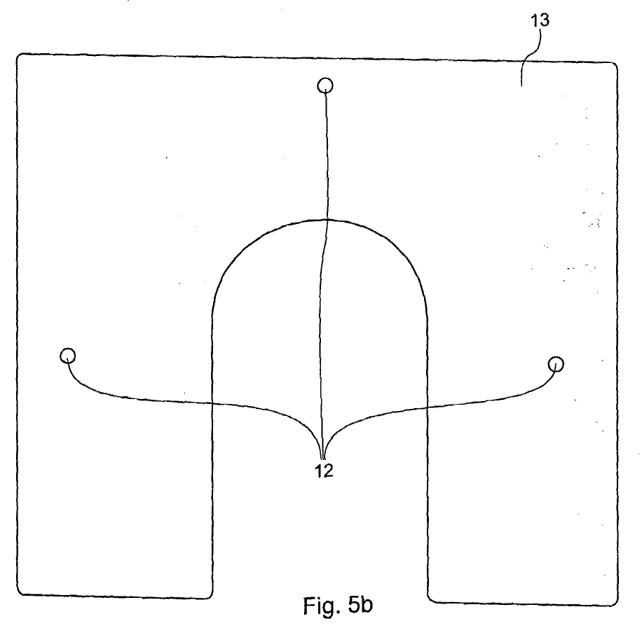

FIG. 5a shows the protective cap with magnets and elongated holes for the fastening of a conventional stoma belt

FIG. 5b shows the magnets at a stoma closing aid as it was published in a parallel application.

### DETAILED DESCRIPTION OF THE FIGURES

FIGS. 1a to 1b show a stoma protective cap 7 on the body of a person. In this case, a collection bag which is connected to the stoma is located below the stoma protective cap. The stoma protective cap is clamped on a belt or a waistband 9.

FIGS. 2a to 2c show a preferred embodiment of the stoma protective cap from various perspectives. It is clearly apparent that the stoma protective cap has a curvature or a curved cap 3 which makes it possible that the pressure which is produced by the clothing on the stoma and the collection bag is diverted and transmitted laterally onto the body. To ensure that the stoma protective cap is held in place, lateral guides 1 are provided through which clothing or the belt or the trouser waistband partially extends. The edge region of the stoma protective cap is formed reinforced, wherein the curved cap is preferably formed thinner and more elastic and is open to the bottom (opening 6) so that the collection bag can escape there.

The guides are formed as elongated hole slots into which the trouser waistband or the belt can be threaded. E.g. belt 10 initially enters under the stoma protective cap in order to protrude to the outside or to the front through the slot in order to then be fed via the cap so that it once again enters through the guide for a short time under the cap.

FIGS. 3a to 3c show a similar concept in which, however, instead of the slots, the guides are formed as clips 5 which are fastened in the upper region on the stoma protective cap and whose lower region is exposed such that clothing can be clamped under this clip. The clip can be formed in one piece from the plate or have been subsequently connected to this e.g. by riveting, gluing or welding.

FIGS. 3d to 3e show the stoma protective cap with a bracket, wherein the bracket is formed at an alternative position so that it can be fastened onto commercially available stoma belts. In this case, the bracket is formed centrally. Elongated holes 11 are also formed shorter and are closed on both sides so that the commercially available stoma belt can be guided through these.

FIG. 4 shows a fastening of a stoma belt on the protective cap. In this case, the stoma belt is guided through the elongated hole and connected to a buckle. The elongated hole is in this case restricted on both sides. Slipping out is thus prevented.

FIG. 5a shows the protective cap with magnets 12 and elongated holes 11 for the fastening of a conventional stoma belt. In this case, corresponding magnets are arranged both on the belt and on the protective cap and thus allow secure holding. It is also conceivable that Velcro closures are used.

FIG. 5b shows the magnets on a stoma closing aid 13 as has been described in a parallel application which was filed on the same day. The content of this application is part of the present application. Due to the identical arrangement of the magnets, these can perform holding functions for different applications.

It is hereby indicated that the stoma protective cap can naturally also be produced from cardboard or a similar pulp product and represents a disposable article which is only used once together with the collection bag.

### LIST OF REFERENCE NUMBERS

1 Guides
2 Upper edge
3 Curved cap
4 Plate
5 Clamping bracket
6 Opening
7 Stoma protective cap
8 Collection bag
9 Belt
10 Belt or waistband
11 Elongated hole for stoma belt
12 Magnets
13 Stoma closing aid

1. Stoma protective cap for protection of the stoma, comprising:
a plate disposable around the stoma, having a curvature, the curvature is formed such that it spans a cavity across the stoma when used, and
the plate encompasses at least one retaining region which enables a fastening on clothing
wherein the retaining region is formed as slot guides which allow that the the stoma protective cap can be slip onto a belt or a trouser waistband so that at least parts of the guide and the curvature are arranged in the state of use between the stoma and the trouser waistband or belt, while the rest of the plate is located at the far side of the trouser waistband or belt.

3. Stoma protective cap according to claim 1, wherein the guides are respectively connected with one end to the plate the guides continue in parallel to the surface of the plate.

3. Stoma protective cap according to claim 1, wherein the guides are slots within the plate located beyond the curvature.

4. Stoma protective cap according to claim 1, wherein the curvature forms an opening in use at the bottom.
